(19) United States
(12) Patent Application Publication
(10) Pub. No.: US 2015/0289939 A1
(43) Pub. Date: Oct. 15, 2015

(54) MAGNETIC DEVICE FOR USE IN AN MPI APPARATUS

(71) Applicant: KONINKLIJKE PHILIPS N.V., Eindhoven (NL)

(72) Inventors: Jügen Erwin Rahmer, Hamburg (DE); Bernhard Gleich, Hamburg (DE); Nils Dennie Nothnagel, Madrid (ES)

(21) Appl. No.: 14/440,235

(22) PCT Filed: Oct. 21, 2013
(86) PCT No.: PCT/IB2013/059490

§ 371 Date: May 1, 2015

Related U.S. Application Data
(60) Provisional application No. 61/723,433, filed on Nov. 7, 2012.

(51) Int. Cl. A61B 19/00; A61B 5/06; A61B 5/00; A61B 5/05

(52) U.S. Cl. CPC A61B 19/22; A61B 5/0515; A61B 5/062; A61B 5/4836; A61B 19/20; A61B 2019/2261; A61B 2562/0223

(57) ABSTRACT

The present invention relates to a magnetic device (400a) that can be localized and moved by a magnetic particle imaging apparatus, said magnetic device comprising a force-receiving portion (410a) formed by one or more ferromagnetic force-receiving elements, which force-receiving portion can be moved and/or oriented by use of magnetic fields, and a localization portion (420a) formed by one or more soft-magnetic localization elements arranged within or at a predetermined distance from said force-receiving portion, which localization portion provides response signals in response to the movement of a substantially field free area of a magnetic field over the location of the localization portion.

### FIELD OF THE INVENTION

The present invention relates to a magnetic device that can be localized and moved by a magnetic particle imaging apparatus. The present invention relates further to an apparatus and method for localizing and moving such a magnetic device.

### BACKGROUND OF THE INVENTION

Magnetic manipulation is a promising approach that enables contact-free manipulation of devices in a patient. Examples are magnetic catheter tips that can be guided into the desired direction or magnetic pills that can locally deliver drug or collect information, such as the magnetically guided capsule endoscopy (MGCE). The approaches enable safer and more comfortable interventional procedures. Existing magnetic manipulation systems, however, require large dedicated field applicators. For instance, Carpi et al., “Controlled navigation of endoscopic capsules: Concept and preliminary experimental investigations”, IEEE Trans. Bio. Med. Eng., vol. 54, no. 11, pp. 2028-2036, November 2007 and “Magnetic Maneuvering of Endoscopic Capsules by Means of a Robotic Navigation System”, IEEE Transactions on biomedical engineering, vol. 56, No. 5, May 2009 describes a wireless capsule endoscopy arranged with magnetic shells for being manipulated and monitored by a robotic magnetic navigation system.

Magnetic Particle Imaging (MPI) is an emerging medical imaging modality. The first versions of MPI were two-dimensional in that they produced two-dimensional images. Newer versions are three-dimensional (3D). A four-dimensional image of a non-static object can be created by combining a temporal sequence of 3D images to a movie, provided the object does not significantly change during the data acquisition for a single 3D image.

MPI is a reconstructive imaging method, like Computed Tomography (CT) or Magnetic Resonance Imaging (MRI). Accordingly, an MP image of an object's volume of interest is generated in two steps. The first step, referred to as data acquisition, is performed using an MPI scanner. The MPI scanner has means to generate a static magnetic gradient field, called the “selection field”, which has a (single) field-free point (FFP) or a field-free line (FFL) at the isocenter of the scanner. Moreover, this FFP (or the FFL; mentioning “FFP” in the following shall generally be understood as meaning FFP or FFL) is surrounded by a first sub-zone with a low magnetic field strength, which is in turn surrounded by a second sub-zone with a higher magnetic field strength. In addition, the scanner has means to generate a time-dependent, spatially nearly homogeneous magnetic field. Actually, this field is obtained by superposing a rapidly changing field with a small amplitude, called the “drive field”, and a slowly varying field with a large amplitude, called the “focus field”. By adding the time-dependent drive and focus fields to the static selection field, the FFP may be moved along a predetermined FFP trajectory throughout a “volume of scanning” surrounding the isocenter. The scanner also has an arrangement of one or more, e.g. three, receive coils and can record any voltages induced in these coils. For the data acquisition, the object to be imaged is placed in the scanner such that the object's volume of interest is enclosed by the scanner's field of view, which is a subset of the volume of scanning.

The object must contain magnetic nanoparticles or other magnetic non-linear materials; if the object is an animal or a patient, a contrast agent containing such particles is administered to the animal or patient prior to the scan. During the data acquisition, the MPI scanner moves the FFP along a deliberately chosen trajectory that traces out/covers the volume of scanning, or at least the field of view. The magnetic nanoparticles within the object experience a changing magnetic field and respond by changing their magnetization. The changing magnetization of the nanoparticles induces a time-dependent voltage in each of the receive coils. This voltage is sampled in a receiver associated with the receive coil. The samples output by the receivers are recorded and constitute the acquired data. The parameters that control the details of the data acquisition make up the “scan protocol”.

In the second step of the image generation, referred to as image reconstruction, the image is computed, or reconstructed, from the data acquired in the first step. The image is a discrete 3D array of data that represents a sampled approximation to the position-dependent concentration of the magnetic nanoparticles in the field of view. The reconstruction is generally performed by a computer, which executes a suitable computer program. Computer and computer program realize a reconstruction algorithm. The reconstruction algorithm is based on a mathematical model of the data acquisition. As with all reconstructive imaging methods, this model can be formulated as an integral operator that acts on the acquired data; the reconstruction algorithm tries to undo, to the extent possible, the action of the model.

Such an MPI apparatus and method have the advantage that they can be used to examine arbitrary examination objects e.g. human bodies in a non-destructive manner and with a high spatial resolution, both close to the surface and remote from the surface of the examination object. Such an apparatus and method are generally known and have been first described in DE 101 51 778 A1 and in Gleich, B. and Weizenecker, J. (2005), “Tomographic imaging using the nonlinear response of magnetic particles” in Nature, vol. 435, pp. 1214-1217, in which also the reconstruction principle is generally described. The apparatus and method for magnetic particle imaging (MPI) described in that publication take advantage of the non-linear magnetization curve of small magnetic particles.

US 2012/0157823 discloses an apparatus for controlling the movement of a catheter through an object and for localizing the catheter within the object, said catheter comprising a magnetic element at or near its tip. The invention applies the principles and hardware of MPI both for catheter localization and catheter movement and provides appropriate control means for controlling the signal generator units to generate and provide control currents to the respective field coils to generate appropriate magnetic fields for moving the catheter through the object in a direction instructed by movement commands and for localizing the catheter within the object.

### SUMMARY OF THE INVENTION

It is an object of the present invention to provide a magnetic device that can be both localized using MPI and manipulated using magnetic forces generated by the magnetic fields applied in the MPI apparatus.

It is a further object of the present invention to provide an apparatus for localizing and moving such a magnetic device.

In an aspect of the present invention a magnetic device is presented that can be localized and moved by a magnetic particle imaging apparatus, said magnetic device comprising:
a force-receiving portion formed by one or more ferromagnetic force-receiving elements, which force-receiving portion can be moved and/or oriented by use of magnetic fields,
a localization portion formed by one or more soft-magnetic localization elements arranged within or at a predetermined distance from said force-receiving portion, which localization portion provides response signals in response to the movement of a substantially field free area of a magnetic field over the location of the localization portion.

Preferred embodiments of the invention are defined in the dependent claims.

Existing magnetic manipulation systems require large dedicated field applicators. In contrast, an MPI apparatus can generate the required fields and field gradients without (substantial) hardware modification and at the same time adds the possibility of real time device localization. The proposed magnetic device comprises a force-receiving portion and an MPI-signal-generating (localization) portion. The force-receiving portion is preferably configured to take up forces and torques from a magnetic gradient field. The localization portion is configured to generate a localization signal (i.e. appropriate detection signals acquired by the MPI apparatus that enable the localization of the magnetic device). Preferably, the force-receiving portion is configured such that it does not interfere or does not prevent a MPI apparatus from detecting the localization signal. This design allows simultaneous or interleaved imaging and manipulation of the magnetic device. Furthermore, by removing the characteristic signal of the magnetic device, MPI of particles in the blood or tissue can be performed simultaneously.

The possibility of applying very strong magnetic field gradients in an MPI apparatus enables the exertion of rather strong forces on magnetic devices. This can for instance be used for guiding autonomous devices like a pill through the gastro-intestinal tract or for guiding the magnetic tip of a catheter.

In an embodiment said localization portion is arranged at a location at which the force receiving portion generates the lowest distortion of the magnetic fields applied for localization of the localization portion. This provides that the detection signals from the localization portion can be obtained with good quality.

Further, in an embodiment said localization portion is arranged in a central area, in particular a symmetry center, within said force-receiving portion. With this arrangement the detection signals from the localization portion are generally obtained in the best quality and are least disturbed by any signal coming from the force receiving portion.

Depending on the particular implementation, in particular the available space and the desired signal accuracy said one or more localization elements comprise one or more soft-magnetic elements in the form of spheres, needles, patches, particles or foils. Different shapes have different orientation-dependent demagnetizing factors. Demagnetization reduces the signal response if the factor is N>0 in a certain direction. A needle delivers high signal only if a field component is aligned with its axis (where N˜0). Thus, its orientation axis can be inferred from the orientation-dependent response. With two orthogonal needles, not only one axis, but the full orientation in space can be determined. A patch has good signal in two directions, which is good for localization, but not so good for orientation determination. A soft-magnetic sphere has an equal demagnetizing factor in all orientations and thus rather low signal. However, harder magnetic material may also deliver good signal, if not annealed.

In an embodiment said one or more localization elements comprise at least two soft-magnetic elements arranged in a non-coplanar orientation with respect to each other. This enables determining the orientation of the magnetic device.

Further, in an embodiment said localization portion further comprises a bearing, in particular a fluid bearing, allowing the one or more localization elements to align with an applied magnetic field. This is particularly of interest in case the orientation of the magnetic device shall be changed, e.g. if the magnetic device is arranged at the tip of a catheter which shall be moved around in a patients body, e.g. the gastrointestinal tract.

Preferably, said one or more force-receiving elements comprise two or more ferromagnetic elements in the form of spheres arranged around said localization portion. This provides for a simply implementable but effective force-receiving portion.

Further, said two or more ferromagnetic elements are arranged at the corners of a highly symmetric body, such as a pyramid, in particular a tetrahedron. This arrangement is still rather simple, but has a certain degree of symmetry that reduces the field distortions at the localization portion.

In an embodiment said one or more force-receiving elements comprise a housing formed of a ferromagnetic material around said localization portion, said housing having a number of openings and/or slits. This embodiment allows easy maneuvering within a body, but still enables that the magnetic field well reaches the localization portion.

Preferably, said one or more force-receiving elements are made of an annealed soft-magnetic material. This provides that the detection signals of the localization portion is not (or at least not much) disturbed.

In another embodiment said force-receiving portion is configured to change its magnetization, in particular to reduce its magnetization when the magnetic device shall be localized. This can, for instance, be achieved by changing the orientation of force-receiving elements having a fixed magnetization direction.

In another implementation said force-receiving portion comprises a switch, in particular an actuator or controller, to change the magnetization of the force-receiving portion.

Still further, in an embodiment said force-receiving portion is made of anisotropic material, is formed in elongated form and/or comprises one or more permanent magnets. This provides that the magnetic device can take up torques in an applied magnetic field.

According to another aspect an apparatus for localizing and moving a magnetic device according to the present invention is presented, which apparatus comprises:
selection means comprising a selection field signal generator unit and selection field elements for generating a magnetic selection field having a pattern in space of its magnetic field strength such that a first sub-zone having a low magnetic field strength where the magnetization of soft-magnetic localization elements of the magnetic device is not saturated and a second sub-zone having a higher magnetic field strength where the magnetization of soft-magnetic localization elements of the magnetic device is saturated are formed in the field of view,
drive means comprising a drive field signal generator unit and drive field coils for changing the position in space of the two sub-zones in the field of view by means of a magnetic drive field so that the magnetization of the soft-magnetic localization elements of the magnetic device changes locally,
focus means for changing the position in space of the field of view,
receiving means comprising at least one signal receiving unit and at least one receiving coil for acquiring detection signals, which detection signals depend on the magnetization in the field of view, which magnetization is influenced by the change in the position in space of the first and second sub-zone,
processing means for processing said detection signals, and
control means for controlling said selection means, said drive means and said focus means to generate magnetic fields to move the field of view to a position such that the magnetic device is positioned between a target position of the magnetic device and the center of the field of view in order to generate a force for moving the magnetic device in the direction of the target position and to thereafter or simultaneously move the field of view to a position such that the magnetic device is positioned within the field of view for localizing the magnetic device.

Preferably, said control means is adapted to control said selection means, said drive means and said focus means to generate magnetic fields to alternately move the field of view into the position for generating a force onto the magnetic device for moving it into the direction of the target position and into the position for localizing the magnetic device until the magnetic device has reached the target position.

### BRIEF DESCRIPTION OF THE DRAWINGS

These and other aspects of the invention will be apparent from and elucidated with reference to the embodiment(s) described hereinafter. In the following drawings

FIG. 1 shows a first embodiment of an MPI apparatus,

FIG. 2 shows an example of the selection field pattern produced by an apparatus as shown in FIG. 1,

FIG. 3 shows a second embodiment of an MPI apparatus,

FIG. 4 shows a third and a fourth embodiment of an MPI apparatus,

FIG. 5 shows a block diagram of an MPI apparatus,

FIG. 6 shows a first embodiment of a magnetic device according to the present invention,

FIG. 7 shows the forces acting on the first embodiment of the magnetic device at different positions within the magnetic selection field,

FIG. 8 shows a second embodiment of a magnetic device according to the present invention,

FIG. 9 shows a third embodiment of a magnetic device according to the present invention,

FIG. 10 shows a fourth embodiment of a magnetic device according to the present invention,

FIG. 11 shows a fifth embodiment of a magnetic device according to the present invention,

FIG. 12 shows a sixth embodiment of a magnetic device according to the present invention,

FIG. 13 shows a seventh embodiment of a magnetic device according to the present invention,

FIG. 14 shows a diagram illustrating a first embodiment of controlling a magnetic device according to the present invention,

FIG. 15 shows a diagram illustrating a second embodiment of controlling a magnetic device according to the present invention

FIG. 16 shows a diagram illustrating a third embodiment of controlling a magnetic device according to the present invention,

FIG. 17 shows a diagram illustrating a particular example of controlling a magnetic device according to the present invention, and

FIG. 18 shows a diagram illustrating another example of controlling a magnetic device according to the present invention.

### DETAILED DESCRIPTION OF THE INVENTION

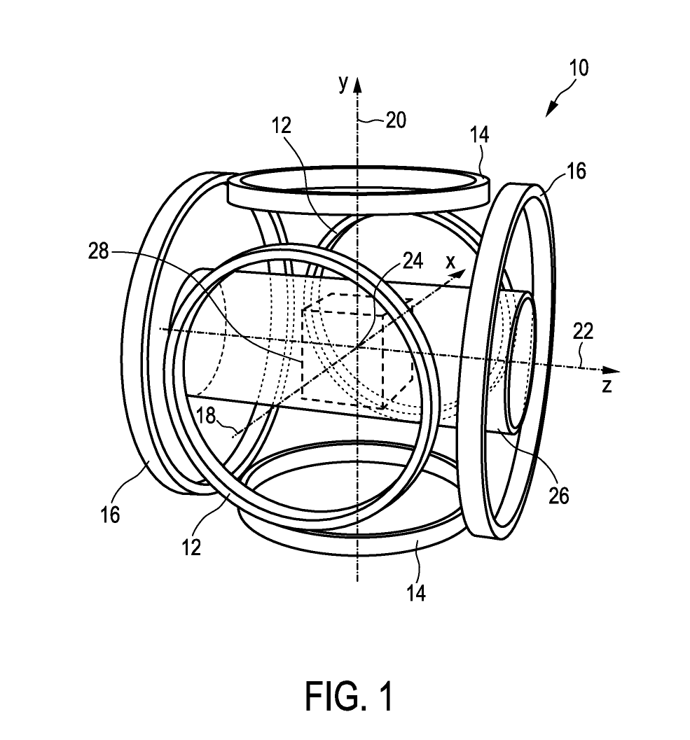

Before the details of the present invention shall be explained, basics of magnetic particle imaging shall be explained in detail with reference to FIGS. 1 to 4. In particular, four embodiments of an MPI scanner for medical diagnostics will be described. An informal description of the data acquisition will also be given. The similarities and differences between the different embodiments will be pointed out. Generally, the present invention can be used in all these different embodiments of an MPI apparatus.

The first embodiment 10 of an MPI scanner shown in FIG. 1 has three pairs 12, 14, 16 of coaxial parallel circular coils, these coil pairs being arranged as illustrated in FIG. 1. These coil pairs 12, 14, 16 serve to generate the selection field as well as the drive and focus fields. The axes 18, 20, 22 of the three coil pairs 12, 14, 16 are mutually orthogonal and meet in a single point, designated the isocenter 24 of the MPI scanner 10. In addition, these axes 18, 20, 22 serve as the axes of a 3D Cartesian x-y-z coordinate system attached to the isocenter 24. The vertical axis 20 is nominated the y-axis, so that the x- and z-axes are horizontal. The coil pairs 12, 14, 16 are named after their axes. For example, the y-coil pair 14 is formed by the coils at the top and the bottom of the scanner. Moreover, the coil with the positive (negative) y-coordinate is called the y′-coil (y-coil), and similarly for the remaining coils. When more convenient, the coordinate axes and the coils shall be labelled with x₁, x₂, and x₃, rather than with x, y, and z.

The scanner 10 can be set to direct a predetermined, time-dependent electric current through each of these coils 12, 14, 16, and in either direction. If the current flows clockwise around a coil when seen along this coil's axis, it will be taken as positive, otherwise as negative. To generate the static selection field, a constant positive current I^{S} is made to flow through the z⁺-coil, and the current −I^{S} is made to flow through the z⁻-coil. The z-coil pair 16 then acts as an anti-parallel circular coil pair.

It should be noted here that the arrangement of the axes and the nomenclature given to the axes in this embodiment is just an example and might also be different in other embodiments. For instance, in practical embodiments the vertical axis is often considered as the z-axis rather than the y-axis as in the present embodiment. This, however, does not generally change the function and operation of the device and the effect of the present invention.

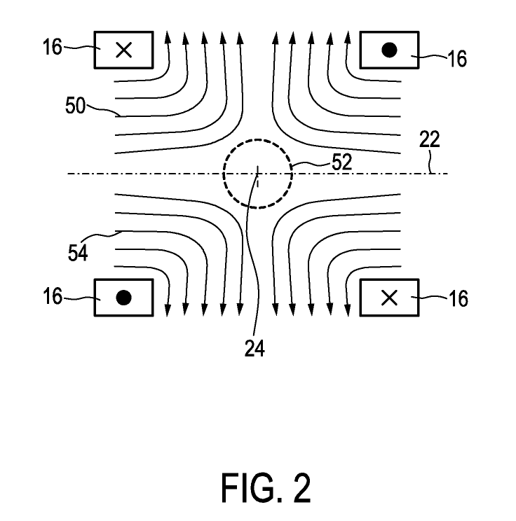

The magnetic selection field, which is generally a magnetic gradient field, is represented in FIG. 2 by the field lines 50. It has a substantially constant gradient in the direction of the (e.g. horizontal) z-axis 22 of the z-coil pair 16 generating the selection field and reaches the value zero in the isocenter 24 on this axis 22. Starting from this field-free point (not individually shown in FIG. 2), the field strength of the magnetic selection field 50 increases in all three spatial directions as the distance increases from the field-free point. In a first sub-zone or region 52 which is denoted by a dashed line around the isocenter 24 the field strength is so small that the magnetization of particles present in that first sub-zone 52 is not saturated, whereas the magnetization of particles present in a second sub-zone 54 (outside the region 52) is in a state of saturation. In the second sub-zone 54 (i.e. in the residual part of the scanner's field of view 28 outside of the first sub-zone 52) the magnetic field strength of the selection field is sufficiently strong to keep the magnetic particles in a state of saturation.

By changing the position of the two sub-zones 52, 54 (including the field-free point) within the field of view 28 the (overall) magnetization in the field of view 28 changes. By determining the magnetization in the field of view 28 or physical parameters influenced by the magnetization, information about the spatial distribution of the magnetic particles in the field of view 28 can be obtained. In order to change the relative spatial position of the two sub-zones 52, 54 (including the field-free point) in the field of view 28, further magnetic fields, i.e. the magnetic drive field, and, if applicable, the magnetic focus field, are superposed to the selection field 50.

To generate the drive field, a time dependent current I^{D}₁ is made to flow through both x-coils 12, a time dependent current I^{D}₂ through both y-coils 14, and a time dependent current I^{D}₃ through both z-coils 16. Thus, each of the three coil pairs acts as a parallel circular coil pair. Similarly, to generate the focus field, a time dependent current I^{F}₁ is made to flow through both x-coils 12, a current I^{F}₂ through both y-coils 14, and a current I^{F}₃ through both z-coils 16.

It should be noted that the z-coil pair 16 is special: It generates not only its share of the drive and focus fields, but also the selection field (of course, in other embodiments, separate coils may be provided). The current flowing through the z^{±}-coil is I^{D}₃+I^{F}₃±I^{S}. The current flowing through the remaining two coil pairs 12, 14 is I^{D}ₖ+I^{F}ₖ, k=1, 2. Because of their geometry and symmetry, the three coil pairs 12, 14, 16 are well decoupled. This is wanted.

Being generated by an anti-parallel circular coil pair, the selection field is rotationally symmetric about the z-axis, and its z-component is nearly linear in z and independent of x and y in a sizeable volume around the isocenter 24. In particular, the selection field has a single field-free point (FFP) at the isocenter. In contrast, the contributions to the drive and focus fields, which are generated by parallel circular coil pairs, are spatially nearly homogeneous in a sizeable volume around the isocenter 24 and parallel to the axis of the respective coil pair. The drive and focus fields jointly generated by all three parallel circular coil pairs are spatially nearly homogeneous and can be given any direction and strength, up to some maximum strength. The drive and focus fields are also time-dependent. The difference between the focus field and the drive field is that the focus field varies slowly in time and may have a large amplitude, while the drive field varies rapidly and has a small amplitude. There are physical and biomedical reasons to treat these fields differently. A rapidly varying field with a large amplitude would be difficult to generate and potentially hazardous to a patient.

In a practical embodiment the FFP can be considered as a mathematical point, at which the magnetic field is assumed to be zero. The magnetic field strength increases with increasing distance from the FFP, wherein the increase rate might be different for different directions (depending e.g. on the particular layout of the device). As long as the magnetic field strength is below the field strength required for bringing magnetic particles into the state of saturation, the particle actively contributes to the signal generation of the signal measured by the device; otherwise, the particles are saturated and do not generate any signal.

The embodiment 10 of the MPI scanner has at least one further pair, preferably three further pairs, of parallel circular coils, again oriented along the x-, y-, and z-axes. These coil pairs, which are not shown in FIG. 1, serve as receive coils. As with the coil pairs 12, 14, 16 for the drive and focus fields, the magnetic field generated by a constant current flowing through one of these receive coil pairs is spatially nearly homogeneous within the field of view and parallel to the axis of the respective coil pair. The receive coils are supposed to be well decoupled. The time-dependent voltage induced in a receive coil is amplified and sampled by a receiver attached to this coil. More precisely, to cope with the enormous dynamic range of this signal, the receiver samples the difference between the received signal and a reference signal. The transfer function of the receiver is non-zero from zero Hertz (“DC”) up to the frequency where the expected signal level drops below the noise level. Alternatively, the MPI scanner has no dedicated receive coils. Instead the drive field transmit coils are used as receive coils.

The embodiment 10 of the MPI scanner shown in FIG. 1 has a cylindrical bore 26 along the z-axis 22, i.e. along the axis of the selection field. All coils are placed outside this bore 26. For the data acquisition, the patient (or object) to be imaged is placed in the bore 26 such that the patient's volume of interest that volume of the patient (or object) that shall be imaged—is enclosed by the scanner's field of view 28 that volume of the scanner whose contents the scanner can image. The patient (or object) is, for instance, placed on a patient table. The field of view 28 is a geometrically simple, isocentric volume in the interior of the bore 26, such as a cube, a ball, a cylinder or an arbitrary shape. A cubical field of view 28 is illustrated in FIG. 1.

The size of the first sub-zone 52 is dependent on the strength of the gradient of the magnetic selection field and on the field strength of the magnetic field required for saturation, which in turn depends on the magnetic particles. For a sufficient saturation of typical magnetic particles at a magnetic field strength of 80 A/m and a gradient (in a given space direction) of the field strength of the magnetic selection field amounting to 50×10³ A/m², the first sub-zone 52 in which the magnetization of the particles is not saturated has dimensions of about 1 mm (in the given space direction).

The patient's volume of interest is supposed to contain magnetic nanoparticles. Prior to the diagnostic imaging of, for example, a tumor, the magnetic particles are brought to the volume of interest, e.g. by means of a liquid comprising the magnetic particles which is injected into the body of the patient (object) or otherwise administered, e.g. orally, to the patient.

Generally, various ways for bringing the magnetic particles into the field of view exist. In particular, in case of a patient into whose body the magnetic particles are to be introduced, the magnetic particles can be administered by use of surgical and non-surgical methods, and there are both methods which require an expert (like a medical practitioner) and methods which do not require an expert, e.g. can be carried out by laypersons or persons of ordinary skill or the patient himself/herself. Among the surgical methods there are potentially non-risky and/or safe routine interventions, e.g. involving an invasive step like an injection of a contrast agent into a blood vessel (if such an injection is at all to be considered as a surgical method), i.e. interventions which do not require considerable professional medical expertise to be carried out and which do not involve serious health risks. Further, non-surgical methods like swallowing or inhalation can be applied.

Generally, the magnetic particles are pre-delivered or pre-administered before the actual steps of data acquisition are carried out. In embodiments, it is, however, also possible that further magnetic particles are delivered/administered into the field of view.

An embodiment of magnetic particles comprises, for example, a spherical substrate, for example, of glass which is provided with a soft-magnetic layer which has a thickness of, for example, 5 nm and consists, for example, of an iron-nickel alloy (for example, Permalloy). This layer may be covered, for example, by means of a coating layer which protects the particle against chemically and/or physically aggressive environments, e.g. acids. The magnetic field strength of the magnetic selection field 50 required for the saturation of the magnetization of such particles is dependent on various parameters, e.g. the diameter of the particles, the used magnetic material for the magnetic layer and other parameters.

In the case of e.g. a diameter of 10 μm with such magnetic particles, a magnetic field of approximately 800 A/m (corresponding approximately to a flux density of 1 mT) is then required, whereas in the case of a diameter of 100 μm a magnetic field of 80 A/m suffices. Even smaller values are obtained when a coating of a material having a lower saturation magnetization is chosen or when the thickness of the layer is reduced.

In practice, magnetic particles commercially available under the trade name Resovist (or similar magnetic particles) are often used, which have a core of magnetic material or are formed as a massive sphere and which have a diameter in the range of nanometers, e.g. 40 or 60 nm.

For further details of the generally usable magnetic particles and particle compositions, the corresponding parts of EP 1304542, WO 2004/091386, WO 2004/091390, WO 2004/091394, WO 2004/091395, WO 2004/091396, WO 2004/091397, WO 2004/091398, WO 2004/091408 are herewith referred to, which are herein incorporated by reference. In these documents more details of the MPI method in general can be found as well.

During the data acquisition, the x-, y-, and z-coil pairs 12, 14, 16 generate a position- and time-dependent magnetic field, the applied field. This is achieved by directing suitable currents through the field generating coils. In effect, the drive and focus fields push the selection field around such that the FFP moves along a preselected FFP trajectory that traces out the volume of scanning—a superset of the field of view. The applied field orientates the magnetic nanoparticles in the patient. As the applied field changes, the resulting magnetization changes too, though it responds nonlinearly to the applied field. The sum of the changing applied field and the changing magnetization induces a time-dependent voltage Vₖ across the terminals of the receive coil pair along the xₖ-axis. The associated receiver converts this voltage to a signal Sₖ, which it processes further.

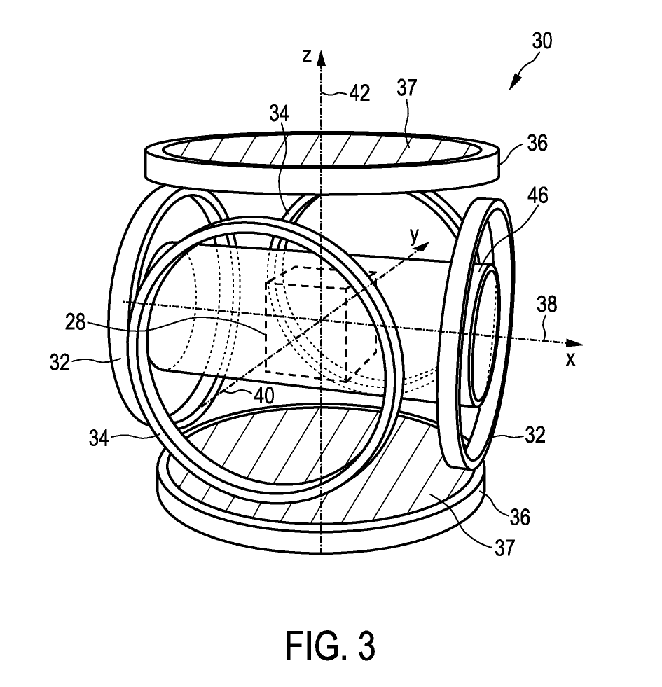

Like the first embodiment 10 shown in FIG. 1, the second embodiment 30 of the MPI scanner shown in FIG. 3 has three circular and mutually orthogonal coil pairs 32, 34, 36, but these coil pairs 32, 34, 36 generate the selection field and the focus field only. The z-coils 36, which again generate the selection field, are filled with ferromagnetic material 37. The z-axis 42 of this embodiment 30 is oriented vertically, while the x- and y-axes 38, 40 are oriented horizontally. The bore 46 of the scanner is parallel to the x-axis 38 and, thus, perpendicular to the axis 42 of the selection field. The drive field is generated by a solenoid (not shown) along the x-axis 38 and by pairs of saddle coils (not shown) along the two remaining axes 40, 42. These coils are wound around a tube which forms the bore. The drive field coils also serve as receive coils.

To give a few typical parameters of such an embodiment: The z-gradient of the selection field, G, has a strength of G/μ₀=2.5 T/m, where μ₀ is the vacuum permeability. The temporal frequency spectrum of the drive field is concentrated in a narrow band around 25 kHz (up to approximately 150 kHz). The useful frequency spectrum of the received signals lies between 50 kHz and 1 MHz (eventually up to approximately 15 MHz). The bore has a diameter of 120 mm. The biggest cube 28 that fits into the bore 46 has an edge length of 120 mm/√2≈84 mm.

Since the construction of field generating coils is generally known in the art, e.g. from the field of magnetic resonance imaging, this subject need not be further elaborated herein.

In an alternative embodiment for the generation of the selection field, permanent magnets (not shown) can be used. In the space between two poles of such (opposing) permanent magnets (not shown) there is formed a magnetic field which is similar to that shown in FIG. 2, that is, when the opposing poles have the same polarity. In another alternative embodiment, the selection field can be generated by a mixture of at least one permanent magnet and at least one coil.

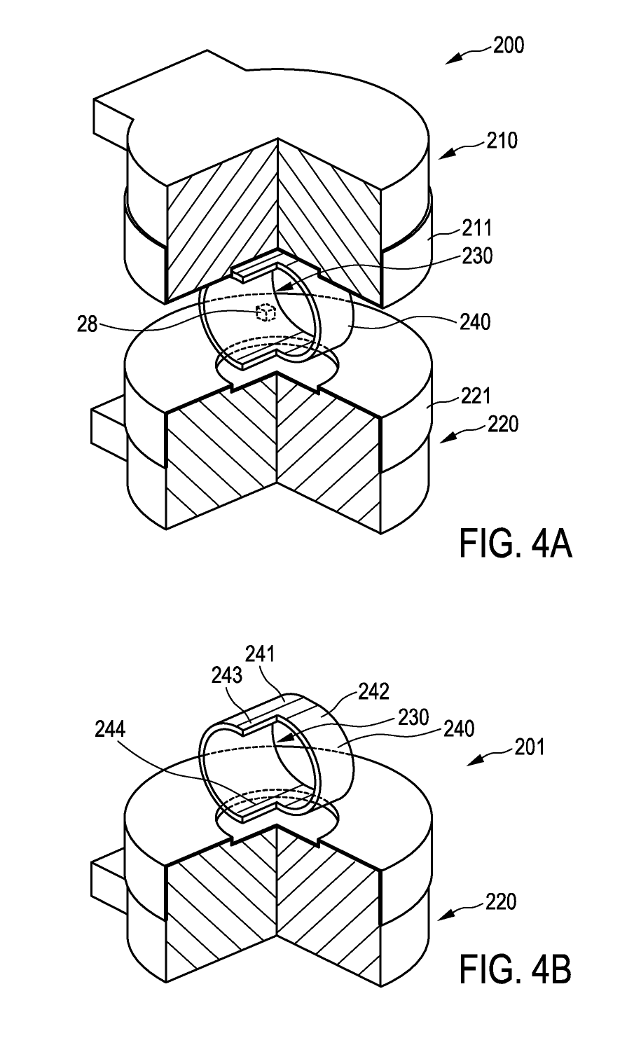

FIG. 4 shows two embodiments of the general outer layout of an MPI apparatus 200, 300. FIG. 4A shows an embodiment of the proposed MPI apparatus 200 comprising two selection-and-focus field coil units 210, 220 which are basically identical and arranged on opposite sides of the examination area 230 formed between them. Further, a drive field coil unit 240 is arranged between the selection-and-focus field coil units 210, 220, which are placed around the area of interest of the patient (not shown). The selection-and-focus field coil units 210, 220 comprise several selection-and-focus field coils for generating a combined magnetic field representing the above-explained magnetic selection field and magnetic focus field. In particular, each selection-and-focus field coil unit 210, 220 comprises a, preferably identical, set of selection-and-focus field coils. Details of said selection-and-focus field coils will be explained below.

The drive field coil unit 240 comprises a number of drive field coils for generating a magnetic drive field. These drive field coils may comprise several pairs of drive field coils, in particular one pair of drive field coils for generating a magnetic field in each of the three directions in space. In an embodiment the drive field coil unit 240 comprises two pairs of saddle coils for two different directions in space and one solenoid coil for generating a magnetic field in the longitudinal axis of the patient.

The selection-and-focus field coil units 210, 220 are generally mounted to a holding unit (not shown) or the wall of room. Preferably, in case the selection-and-focus field coil units 210, 220 comprise pole shoes for carrying the respective coils, the holding unit does not only mechanically hold the selection-and-focus field coil unit 210, 220 but also provides a path for the magnetic flux that connects the pole shoes of the two selection-and-focus field coil units 210, 220.

As shown in FIG. 4a, the two selection-and-focus field coil units 210, 220 each include a shielding layer 211, 221 for shielding the selection-and-focus field coils from magnetic fields generated by the drive field coils of the drive field coil unit 240.

In the embodiment of the MPI apparatus 201 shown in FIG. 4B only a single selection-and-focus field coil unit 220 is provided as well as the drive field coil unit 240. Generally, a single selection-and-focus field coil unit is sufficient for generating the required combined magnetic selection and focus field. Said single selection-and-focus field coil unit 220 may thus be integrated into a (not shown) patient table on which a patient is placed for the examination. Preferably, the drive field coils of the drive field coil unit 240 may be arranged around the patient's body already in advance, e.g. as flexible coil elements. In another implementation, the drive field coil unit 240 can be opened, e.g. separable into two subunits 241, 242 as indicated by the separation lines 243, 244 shown in FIG. 4b in axial direction, so that the patient can be placed in between and the drive field coil subunits 241, 242 can then be coupled together.

In still further embodiments of the MPI apparatus, even more selection-and-focus field coil units may be provided which are preferably arranged according to a uniform distribution around the examination area 230. However, the more selection-and-focus field coil units are used, the more will the accessibility of the examination area for placing a patient therein and for accessing the patient itself during an examination by medical assistance or doctors be limited.

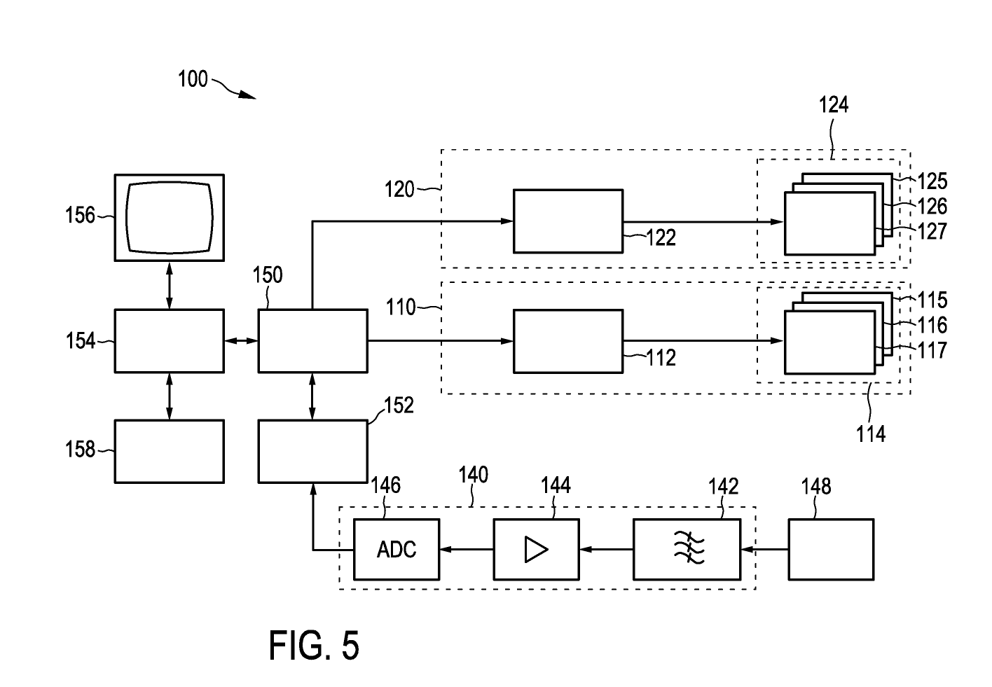

FIG. 5 shows a general block diagram of an MPI apparatus 100 according to the present invention. The general principles of magnetic particle imaging explained above are valid and applicable to this embodiment as well, unless otherwise specified.

The embodiment of the apparatus 100 shown in FIG. 5 comprises various coils for generating the desired magnetic fields. First, the coils and their functions in MPI shall be explained.

For generating the combined magnetic selection-and-focus field, selection-and-focus means 110 are provided. The magnetic selection-and-focus field has a pattern in space of its magnetic field strength such that the first sub-zone (52 in FIG. 2) having a low magnetic field strength where the magnetization of the magnetic particles is not saturated and a second sub-zone (54 in FIG. 4) having a higher magnetic field strength where the magnetization of the magnetic particles is saturated are formed in the field of view 28, which is a small part of the examination area 230, which is conventionally achieved by use of the magnetic selection field. Further, by use of the magnetic selection-and-focus field the position in space of the field of view 28 within the examination area 230 can be changed, as conventionally done by use of the magnetic focus field.

The selection-and-focus means 110 comprises at least one set of selection-and-focus field coils 114 and a selection-and-focus field generator unit 112 for generating selection-and-focus field currents to be provided to said at least one set of selection-and-focus field coils 114 (representing one of the selection-and-focus field coil units 210, 220 shown in FIGS. 4A, 4B) for controlling the generation of said magnetic selection-and-focus field. Preferably, a separate generator subunit is provided for each coil element (or each pair of coil elements) of the at least one set of selection-and-focus field coils 114. Said selection-and-focus field generator unit 112 comprises a controllable current source (generally including an amplifier) and a filter unit which provide the respective coil element with the field current to individually set the gradient strength and field strength of the contribution of each coil to the magnetic selection-and-focus field. It shall be noted that the filter unit 114 can also be omitted. Further, separate focus and selection means are provided in other embodiments.

For generating the magnetic drive field the apparatus 100 further comprises drive means 120 comprising a drive field signal generator unit 122 and a set of drive field coils 124 (representing the drive coil unit 240 shown in FIGS. 4A, 4B) for changing the position in space and/or size of the two sub-zones in the field of view by means of a magnetic drive field so that the magnetization of the magnetic material changes locally. As mentioned above said drive field coils 124 preferably comprise two pairs 125, 126 of oppositely arranged saddle coils and one solenoid coil 127. Other implementations, e.g. three pairs of coil elements, are also possible.

The drive field signal generator unit 122 preferably comprises a separate drive field signal generation subunit for each coil element (or at least each pair of coil elements) of said set of drive field coils 124. Said drive field signal generator unit 122 preferably comprises a drive field current source (preferably including a current amplifier) and a filter unit (which may also be omitted with the present invention) for providing a time-dependent drive field current to the respective drive field coil.

The selection-and-focus field signal generator unit 112 and the drive field signal generator unit 122 are preferably controlled by a control unit 150, which preferably controls the selection-and-focus field signal generator unit 112 such that the sum of the field strengths and the sum of the gradient strengths of all spatial points of the selection field is set at a predefined level. For this purpose the control unit 150 can also be provided with control instructions by a user according to the desired application of the MPI apparatus, which, however, is preferably omitted according to the present invention.

For using the MPI apparatus 100 for determining the spatial distribution of the magnetic particles in the examination area (or a region of interest in the examination area), particularly to obtain images of said region of interest, signal detection receiving means 148, in particular a receiving coil, and a signal receiving unit 140, which receives signals detected by said receiving means 148, are provided. Preferably, three receiving coils 148 and three receiving units 140—one per receiving coil—are provided in practice, but more than three receiving coils and receiving units can be also used, in which case the acquired detection signals are not 3-dimensional but K-dimensional, with K being the number of receiving coils.

Said signal receiving unit 140 comprises a filter unit 142 for filtering the received detection signals. The aim of this filtering is to separate measured values, which are caused by the magnetization in the examination area which is influenced by the change in position of the two part-regions (52, 54), from other, interfering signals. To this end, the filter unit 142 may be designed for example such that signals which have temporal frequencies that are smaller than the temporal frequencies with which the receiving coil 148 is operated, or smaller than twice these temporal frequencies, do not pass the filter unit 142. The signals are then transmitted via an amplifier unit 144 to an analog/digital converter 146 (ADC).

The digitalized signals produced by the analog/digital converter 146 are fed to an image processing unit (also called reconstruction means) 152, which reconstructs the spatial distribution of the magnetic particles from these signals and the respective position which the first part-region 52 of the first magnetic field in the examination area assumed during receipt of the respective signal and which the image processing unit 152 obtains from the control unit 150. The reconstructed spatial distribution of the magnetic particles is finally transmitted via the control means 150 to a computer 154, which displays it on a monitor 156. Thus, an image can be displayed showing the distribution of magnetic particles in the field of view of the examination area.

In other applications of the MPI apparatus 100, e.g. for influencing the magnetic particles (for instance for a hyperthermia treatment) or for moving the magnetic particles (e.g. attached to a catheter for moving the catheter or attached to a medicament for moving the medicament to a certain location) the receiving means may also be omitted or simply not used.

Further, an input unit 158 may optionally be provided, for example a keyboard. A user may therefore be able to set the desired direction of the highest resolution and in turn receives the respective image of the region of action on the monitor 156. If the critical direction, in which the highest resolution is needed, deviates from the direction set first by the user, the user can still vary the direction manually in order to produce a further image with an improved imaging resolution. This resolution improvement process can also be operated automatically by the control unit 150 and the computer 154. The control unit 150 in this embodiment sets the gradient field in a first direction which is automatically estimated or set as start value by the user. The direction of the gradient field is then varied stepwise until the resolution of the thereby received images, which are compared by the computer 154, is maximal, respectively not improved anymore. The most critical direction can therefore be found respectively adapted automatically in order to receive the highest possible resolution.

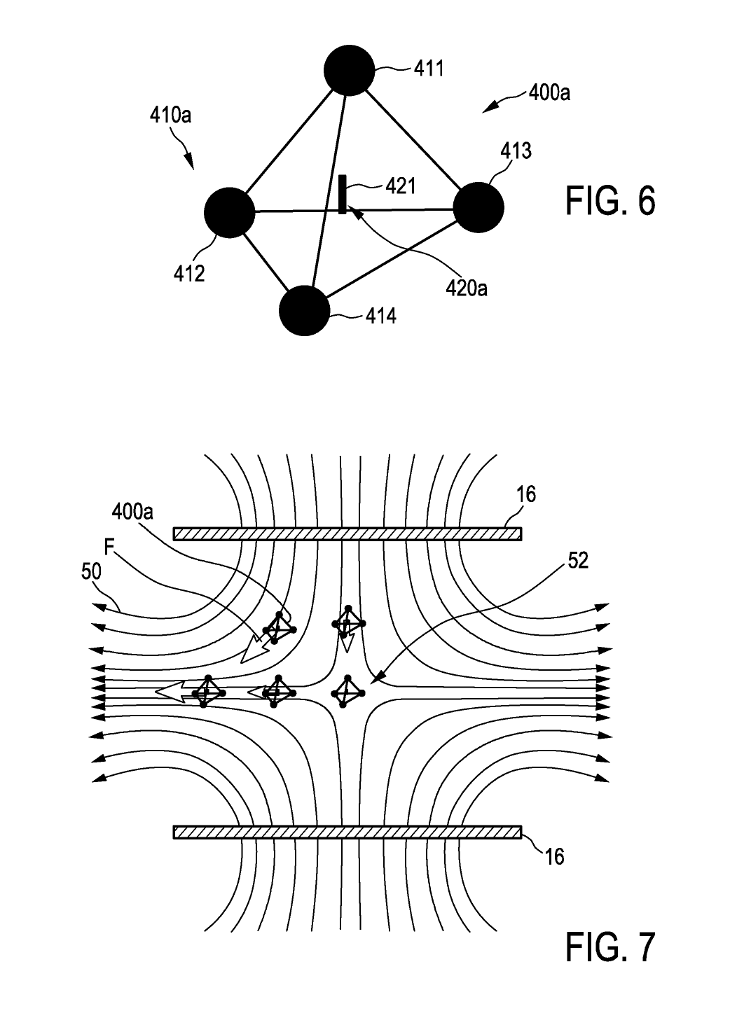

FIG. 6 shows a first embodiment of a magnetic device 400a that can be localized and moved by an MPI apparatus as explained above (or any other embodiment of an MPI apparatus). It comprises a force-receiving portion 410a and a localization portion 420a.

The force-receiving portion 410a is configured such that it can be moved by use of magnetic gradient fields and/or oriented by use of magnetic fields. Generally, it is formed by one or more ferromagnetic force-receiving elements. In this embodiment the force-receiving portion 410a comprises four ferromagnetic spheres 411, 412, 413, 414 that are arranged at the corners of a tetrahedron so that it efficiently takes up forces but does not (or at least not substantially) generate detection signals so that the signal of the localization portion 420a is not (or at least not substantially) disturbed.

The localization portion 420a is configured such that it generates response signals (detection signals) in response to the movement of the substantially field free point (or, more generally, the substantially field free area, which can e.g. have the shape of a line or generally any arbitrary shape) of the magnetic field (generated by the MPI apparatus) over the location of the localization portion 420a. The localization portion 420a is generally formed by one or more soft-magnetic localization elements arranged within said force-receiving portion 410a (or at a predetermined distance from said force-receiving portion). In this embodiment the localization portion 420a comprises a soft-magnetic foil 421 that is arranged in the symmetry center of the force-receiving portion 410a, i.e. in the center of the tetrahedron formed by the ferromagnetic spheres 411, 412, 413, 414. The soft-magnetic foil 421 creates a very strong and sharp response when the field-free point passes over it. The shape of the soft magnetic material is preferably optimized as to reduce shape-induced demagnetization. In an implementation it comprises the shape of a needle which gives good response signal.

FIG. 7 shows the magnetic device 410a at different positions of a magnetic selection field indicated by magnetic field lines 50 (as also shown in FIG. 2) generated by selection coils 16. Arrows F indicates the magnetization vector of the magnetic device 400a, which is generally proportional and parallel to the magnetic field. The magnetic force, which is proportional to the magnitude of the magnetization and points away from the field free area 52, in particular the strength and the direction of the force, applied on the magnetic device 400a are thus dependent on the position of the magnetic device 400a. As can be seen in the substantially field free area 52 (i.e. the first sub-zone) no magnetization and therefore no force is substantially applied on the magnetic device, so that by changing the position of the field free area with respect to the magnetic device, e.g. by using focus fields, the magnetization and thus the force can be adjusted.

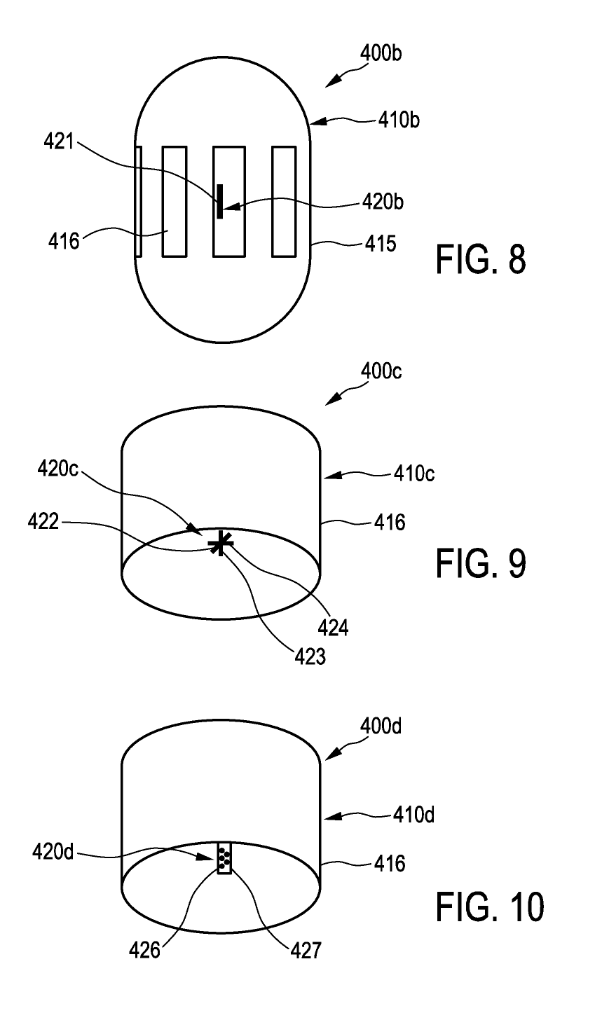

FIG. 8 shows a second embodiment of a magnetic device 400b comprising a force-receiving portion 410b and a localization portion 420b. The force-receiving portion 410b comprises a single part that is configured as an elongated housing (or shell) 415 made of a ferromagnetic material and having the shape of a pill. The housing 415 has slits 416 so that the magnetic field applied by the MPI apparatus can penetrate to the localization portion 420b. The localization portion 420b is arranged inside the housing 415 and may be identically formed as the localization portion 420a.

FIG. 9 shows a third embodiment of a magnetic device 400c comprising a force-receiving portion 410c and a localization portion 420c. The force-receiving portion 410c comprises a single part that is formed as a housing (or shell) 416 made of a ferromagnetic material and having the shape of a disk that is open on the top and/or bottom side. The localization portion 420c is arranged inside the housing 416 and may be identically formed as the localization portion 420a, but in this embodiment it comprises several soft-magnetic needles or patches 422, 423, 424 that are combined in a non-co-planar way (preferably orthogonally) to obtain a good detection signal in all orientations of the FFP trajectory. It shall be noted that those different and/or additional elements of the localization portion 420c may also be used in other embodiments as illustrated above.

FIG. 10 shows a fourth embodiment of a magnetic device 400d comprising a force-receiving portion 410d and a localization portion 420d. The localization portion 420d comprises one or more magnetic nanoparticles 426, e.g. housed in a housing 427, that can be imaged using standard MPI.

Generally, the one or more localization elements of the localization portion are placed at a position where the force-receiving portion generates only a small field distortion. The force-receiving portion generally has a compact or spherical shape and is thus optimized to efficiently take up forces. Further, the force-receiving portion is preferably made of an annealed soft-magnetic material and is thus optimized not to generate detection signals so that the detection signal of the localization portion is not (or at least not much) disturbed.

In another embodiment the force-receiving portion uses a strong anisotropic material and/or is formed as an elongated device to generate a large dipole field. Further, permanent magnets can be assembled. Thus the orientation of the force-receiving portion can be governed (at least in two degrees of freedom) by homogeneous magnetic fields, while the strength of the force is governed by the gradient strength and the distance to the field-free point, which determines the magnitude of magnetization. Permanent magnets have a fixed relationship between direction of magnetization and material and can thus absorb a torque. This embodiment can thus enables the change of the orientation of the magnetic device by use of a torque, which can be exploited in applications in which e.g. a pill comprising a camera shall be oriented with a patient's body, e.g. within the gastrointestinal tract.

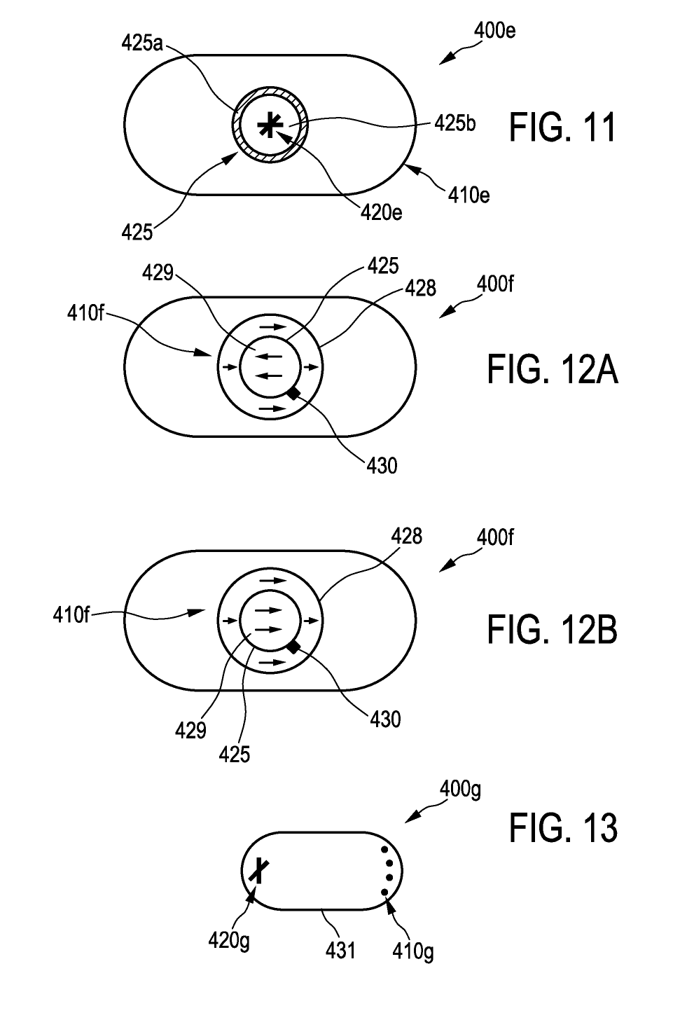

Another embodiment of a magnetic device 400e in the form of a pill is shown in FIG. 11. In this embodiment a bearing 425 is provided as part of the localization portion 420e, which substantially corresponds to the localization portion 420c shown in FIG. 9. The magnetic device 400e further comprises a housing 410e, acting as force receiving portion or containing a separate (not shown) force receiving portion, and enclosing said bearing 425 and said localization portion 420e. The bearing 425 provides that the localization portion 420e can align with the external magnet field independent of the orientation of the force-receiving portion 410e.

The bearing 425 is, in this embodiment, a fluid bearing (e.g. comparable to a liquid compass) comprising a housing 425a enclosing the needles or patches of the localization portion 420e swimming in a liquid 425b filled into said housing 425a so that they can move independently from the housing 425a and the rest of the magnetic device 400e. The bearing 425 preferably has some anisotropy in the localization portion 420e, so that a torque arises that aligns the localization portion 420e with the magnetic field.

For improved imaging, a switchable magnetic device 400f, as shown in FIG. 12, can be conceived, which reduces its magnetization during an imaging sequence that is not related to the device manipulation. Switching using a dedicated field sequence or via internal electronics and/or actuators to rearrange the configuration of the magnetic material in the magnetic device is conceivable. The embodiment of the magnetic device 400f comprises the force receiving portion 410f including an arrangement of two permanent magnets 428, 429 and a switching element 430 formed e.g. by a heat-sensitive bolt. The localization portion is not shown in FIG. 12 and may be located at the other end of the magnetic device 400f or inside the switchable magnet arrangement, which then, however, would have to be slit.

The permanent magnet 428 is surrounding the permanent magnet 429, and the permanent magnets 428, 429 can be independently moved relative to each other. For instance, in this embodiment the permanent magnet 429 is included in a bearing 425 as shown above in FIG. 11.

For instance, by use of strong external magnetic fields the relative orientation of the permanent magnets 428, can be changed from an annular or anti-parallel orientation (which is energetically preferred at low field and provides a low total dipole moment) as shown in FIG. 12A to a parallel orientation (providing a high dipole moment) as shown in FIG. 12B and indicated by the arrows. The desired relative orientation can thus be controlled by the application or absence of strong magnetic fields as long as the permanent magnets can change their orientation with respect to each other (i.e. in case of suitable bearing of the permanent magnets). For switching between the different states shown in FIG. 12 the connection (i.e. the bolt 430 that is e.g. formed by bi-metal) is heated by alternating magnetic fields so that it deblocks the inner magnet 429. Without external magnetic field the magnets 428, 429 can be fixed in the actual orientation by the bolt 430. Thus, the magnetic device 400f is switchable by use of external magnetic fields.

In still another embodiment (not shown) requiring no mechanical elements for switching, the permanent magnets can be heated by use of alternating magnetic fields above the Curie temperature in order to “switch it off”. If it cools down thereafter without magnetic field applied, it will have a lower total magnetization thereafter. Alternatively, the sub-grid magnetization in ferrimagnets can be influenced by application of heat in order to influence the total magnetization.

Still another embodiment of a magnetic device 400g is shown in FIG. 13. In this embodiment the force receiving portion 410g and the localization portion 420g are arranged at a distance, in particular at opposite ends of a housing 431 of the magnetic device 400g. The idea is that when having a rather large device (e.g. a large pill or a catheter), such an arrangement may be simpler and more useful. In particular, mutual disturbance between the portions 410g, 420g is minimized without having to build a complicated symmetric arrangement.

There are various options available for control of the MPI apparatus to both localize the magnetic device and manipulate it using magnetic forces generated by the magnetic fields applied by the MPI apparatus.

For localization time based gridding localization may be applied. Such a gridding algorithm is used to directly generate an image of the field of view from detection signals without using a (generally used) system function. The signal is written in the time domain at the current location of the FFP. A very sharp time signal is obtained that allows an SNR improvement by high-pass filtering resulting in a bright pixel in the image at the position of the localization portion, e.g. a needle made of soft-magnetic material.

For fast localization a threshold plus an average over the center of the mass of several FFP passes may be applied. In this way the bright pixel of the localization portion can be separated from the remaining signal. If, except for the soft-magnetic material, no other material resulting in a detection signal, is within the field of view, the mass center of the signal over the image can be determined to determine the location of the soft-magnetic material.

The localization portion (e.g. the magnetic foil) response depends on the orientation with respect to the drive field. The foil orientation can be determined from the instantaneous response to different FFP trajectory orientations (different peak amplitudes and widths; trajectory containing different directions). This allows determination of the error in localization depending on direction, pre-selection of sharpest peak using knowledge about trajectory and foil orientation and/or determination of the device orientation.

Alternatively, if a 3D drive field sequence is used, the demagnetization effect (e.g. of a needle as localization portion) can be used to determine the orientation of the (localization part of the) device in the field. A needle only is magnetized along is axis. The signal averaged over one trajectory cycle or the signal content in the spectrum is proportional to the projection of the magnetization on the coil axes. For this purpose different FFP trajectories can be used which can efficiently detect this direction dependency, e.g. by quickly re-orienting the FFP trajectory in order to detect orthogonal directions. Alternatively, the trajectory can always be oriented so that the strongest signal is obtained.

Still further, multi-color MPI can be used according to which different materials resulting in detection signals can be detected simultaneously. The obtained signals are separated during reconstruction so that two images are generated which can be combined again in different colors. If multi-color MPI is used time-based removal of peaks, e.g. after localization, allows undisturbed reconstruction of the detection signals, e.g. resulting from particles in blood or tissue.

For manipulation an interleaved mode may be used providing a temporal separation between device localization and force exertion. In a simultaneous mode imaging and force exertion are combined, however the magnetic device must always be kept within the drive field patch (i.e. the field of view).

In an embodiment an orthoview or 3D visualization of the device position is provided. Further, the desired position of the magnetic position can be controlled, e.g. by a joystick or other user interface. For instance, a desired direction pointed at by a pointing device can be automatically translated in adequate field sequences (as described above, by positioning the field-free area at a certain distance opposite to the desired direction) with the effect of pushing the device or catheter tip in the desired direction. Field sequences that shake or rotate the device can help get free it when being stuck. These may be activated by the operator when he has the impression that the device is stuck. Also they might be used to perforate tissue. Force control can be achieved via the distance of the FFP to the object, which determines the degree of magnetization.

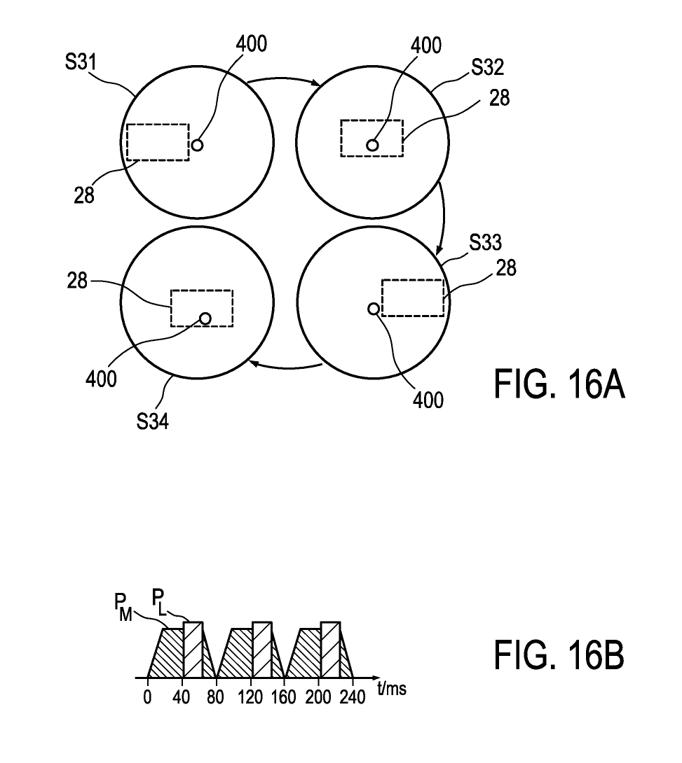

FIG. 14 shows a diagram illustrating a first embodiment of controlling a magnetic device according to the present invention. FIG. 14A shows the steps of the proposed control method. In the beginning (step S10) it will be assumed that the magnetic device 400 is within the field of view (FOV) 28. This can be checked by use of the average signal. If the signal is too low, it is assumed that the magnetic device 400 is not within the FOV 28 and a search routine will be carried out as explained below with reference to FIG. 16.

For exerting a force onto the magnetic device 400, the FOV 28 is briefly moved to a different position (step S11), such that the average FFP position magnetic device 400 is arranged in between the FOV 28 and the target position 500. The FFP motion induced by the drive fields is too fast to lead to a displacement of the magnetic device 400. Therefore, for exerting a force, the average FFP position (averaged over one drive field trajectory) is relevant. This position is changed using the focus fields.

The force receiving portion of the magnetic device 400 is thus reversed in magnetization and is accelerated via the magnetic field gradient away from the FOV 28 towards the target position 500. The force always points in the direction of the highest (absolute) field gradient that is always away from the FFP. Therefore, when the FFP is put on one side of the magnetic device 400, the magnetic device 400 is moved to the opposite side. The distance between the average FFP and the magnetic device 400 determines the magnetization of the magnetic device and, hence, the strength of the force. In the subsequent step S12, the FOV 28 is moved back to a position at which the magnetic device 400 has been localized previously. Then, the loop of steps starts again.

As shown in FIG. 14B, magnetic localization pulses P_{L} for localization of the magnetic device 400 and magnetic force exerting pulses P_{F} for exerting a force on the magnetic device 400 are alternately applied. In this context it shall be noted that, in reality, the localization sequence is not pulsed, but just keeps on running. However, during focus field variations, the signal may be disturbed. Thus, the localization pulses P_{L} correspond to time windows, during which the localization signal is undisturbed and can be evaluated.

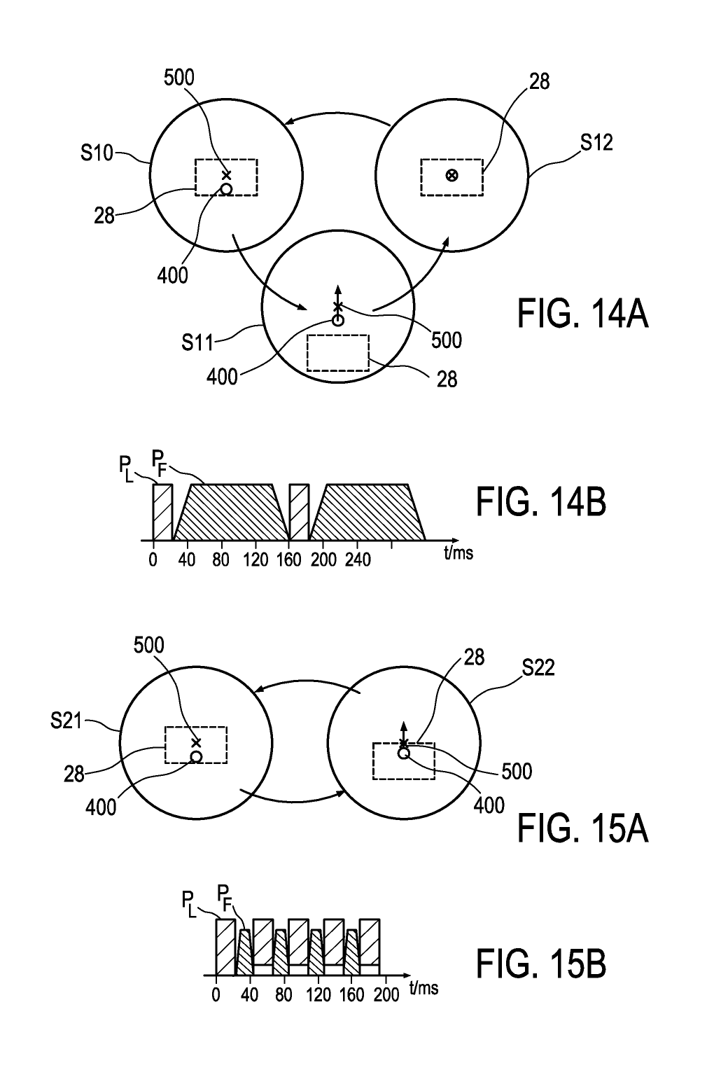

FIG. 15 shows a diagram illustrating a second embodiment of controlling a magnetic device according to the present invention. FIG. 15A shows the steps of this embodiment of the control method. In this embodiment, dead times are avoided occurring in the method explained with reference to FIG. 14 since the FOV 28 is moved to a position too far away from the magnetic device 400 in step S11. According to this embodiment, in a first step S21 the magnetic device 400 is localized. In a subsequent step S22, the FOV 28 is moved to a different position, but not as far as in step S11. The FOV 28 is only positioned such that the magnetic device 400 is located at the border of the FOV 28 and can still be localized. Thus, in this embodiment, localization and force exertion for movement of the magnetic device can be done at the same time as illustrated in FIG. 15B. The disadvantage, however, is that the force that can be applied is lower, since the force increases with increasing distance between the FOV 28 and the magnetic device 400. However, with this embodiment the magnetic device 400 can be localized more often or even all the time, so that the positioning of the magnetic device 400 can be controlled better and faster.

FIG. 16 shows a diagram illustrating a third embodiment of controlling a magnetic device according to the present invention. FIG. 16A shows the steps of this embodiment of the control method. With this embodiment, the magnetic device 400 can be initially searched. For this purpose, the FOV 28 is subsequently moved to different positions as shown in steps S31, S32, S33. The obtained average signal intensity is then compared. The position at which the magnetic device 400 is located generates the strongest signal since the FFP moves directly through the localization portion and changes its magnetization. By this change of magnetization, a voltage signal is induced into the receiving coil. After the initial search, the FOV 28 will be positioned at the position resulting in the strongest average signal, so that thereafter the “real” control can be started as indicated in step S34, e.g. by performing a control method as explained above with reference to FIG. 14 or 15. FIG. 16B shows the movements of the FOV 28 by use of magnetic movement pulses P_{M} applied by focus field means (e.g. focus field coils or select-and-focus field coils) and the localization of the magnetic device 400 by use of magnetic localization pulses P_{L}.

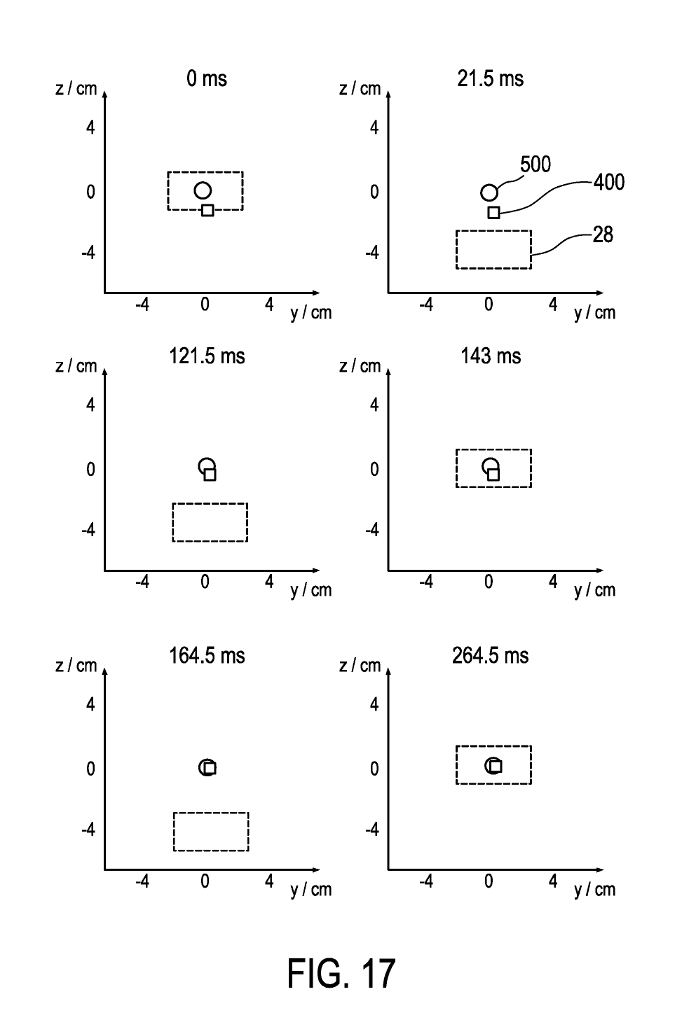

FIG. 17 shows a diagram illustrating a particular example of controlling a magnetic device according to the present invention. After the initial search for the magnetic device 400, the manipulation is started. It can be seen that the magnetic device is located a short distance below the target position 500. In order to correct the position, the FOV 28 is moved for a certain time duration, so that the average FFP is arranged at a larger distance below that target position 500. The FOV 28 remains at this position for a certain time during which the force receiving portion is changed in magnetization and the magnet field gradient exerts a force moving the magnetic device into the direction of the target position 500. Subsequently, the FOV 28 is moved above the magnetic device 400 and the position of the magnetic device 400 is again localized. Since the magnetic device 400 is now closer to the target position, the FOV 28 is moved away again to further force the magnetic device 400 into the direction of the target position 500. Finally, the magnetic device 400 is localized at the target position 500, so that its position can be stabilized.

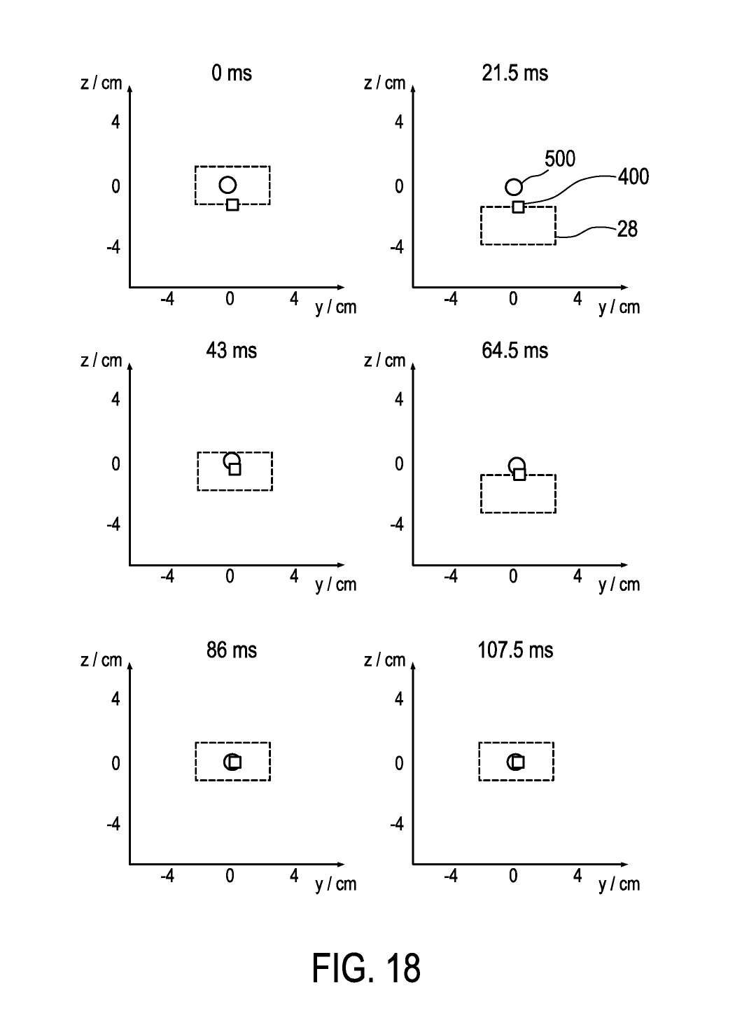

FIG. 18 shows a diagram illustrating another example of controlling a magnetic device according to the present invention. In order to localize more often and without interruptions, the FOV 28 is moved only away from the magnetic device so far that the magnetic device 400 is still covered by the FOV 28. Thus, the magnetic device 400 can still be localized at this position. This is subsequently performed several times until the magnetic device 400 is arranged at the target position 500. In particular, by moving the FOV it is tried to keep the moving device constantly close to the edge of the FOV, to exert a constant force until the target position is reached.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. A magnetic device that can be localized and moved by a magnetic particle imaging apparatus, said magnetic device comprising:
a force-receiving portion formed by one or more ferromagnetic force-receiving elements, which force-receiving portion is arranged to be moved and/or oriented by use of magnetic fields and to move the magnetic device accordingly,
a localization portion formed by one or more soft-magnetic localization elements arranged within or at a predetermined distance from said force-receiving portion, which localization portion is arranged to provides response signals in response to the movement of a substantially field free area of a magnetic field over the location of the localization portion.

2. The magnetic device as claimed in claim 1,
wherein said localization portion is arranged at a location at which the force receiving portion generates the lowest distortion of the magnetic fields applied for localization of the localization portion.

3. The magnetic device as claimed in claim 1,
wherein said localization portion is arranged in a central area, in particular a symmetry center, within said force-receiving portion.

4. The magnetic device as claimed in claim 1,
wherein said one or more localization elements comprise one or more soft-magnetic elements in the form of spheres, needles, patches, particles or foils.

5. The magnetic device as claimed in claim 1,
wherein said one or more localization elements comprise at least two soft-magnetic elements arranged in a non-coplanar orientation with respect to each other.

6. The magnetic device as claimed in claim 1,
wherein said localization portion further comprises a bearing, in particular a fluid bearing, allowing the one or more localization elements to align with a applied magnetic field.

7. The magnetic device as claimed in claim 1,
wherein said one or more force-receiving elements comprise two or more ferromagnetic elements in the form of spheres arranged around said localization portion.

8. The magnetic device as claimed in claim 7,
wherein said two or more ferromagnetic elements are arranged at the corners of a pyramid, in particular of a tetrahedron.

9. The magnetic device as claimed in claim 1,
wherein said one or more force-receiving elements comprise a housing formed of a ferromagnetic material around said localization portion, said housing having a number of openings and/or slits.

10. The magnetic device as claimed in claim 1,
wherein said one or more force-receiving elements are made of an annealed soft-magnetic material.

11. The magnetic device as claimed in claim 1,
wherein said force-receiving portion is configured to change its magnetization, in particular configured to reduce its magnetization when the magnetic device shall be localized.

12. The magnetic device as claimed in claim 11,
wherein said force-receiving portion comprises a switch, in particular an actuator or controller, to change the magnetization of the force-receiving portion.

13. The magnetic device as claimed in claim 1,
wherein said force-receiving portion is made of anisotropic material, is formed in elongated form and/or comprises one or more permanent magnets.

14. An apparatus for localizing and moving a magnetic device as claimed in claim 13, which apparatus comprises:
selection means comprising a selection field signal generator unit and selection field elements for generating a magnetic selection field having a pattern in space of its magnetic field strength such that a first sub-zone having a low magnetic field strength where the magnetization of soft-magnetic localization elements of the magnetic device is not saturated and a second sub-zone having a higher magnetic field strength where the magnetization of soft-magnetic localization elements of the magnetic device is saturated are formed in the field of view,
drive means comprising a drive field signal generator unit and drive field coils for changing the position in space of the two sub-zones in the field of view by means of a magnetic drive field so that the magnetization of the soft-magnetic localization elements of the magnetic device changes locally,
focus means for changing the position in space of the field of view,
receiving means comprising at least one signal receiving unit and at least one receiving coil for acquiring detection signals, which detection signals depend on the magnetization in the field of view, which magnetization is influenced by the change in the position in space of the first and second sub-zone,
processing means for processing said detection signals, and
control means for controlling said selection means, said drive means and said focus means to generate magnetic fields to move the field of view to a position such that the magnetic device is positioned between a target position of the magnetic device and the center of the field of view in order to generate a force for moving the magnetic device in the direction of the target position and to thereafter or simultaneously move the field of view to a position such that the magnetic device is positioned within the field of view for localizing the magnetic device.

15. The apparatus as claimed in claim 14,
wherein said control means is adapted to control said selection means, said drive means and said focus means to generate magnetic fields to alternately move the field of view into the position for generating a force onto the magnetic device for moving it into the direction of the target position and into the position for localizing the magnetic device until the magnetic device has reached the target position.

16. A method for localizing and moving a magnetic device as claimed in claim 13, which method comprises:
generating a magnetic selection fields having a pattern in space of its magnetic field strength such that a first sub-zone having a low magnetic field strength where the magnetization of soft-magnetic localization elements of the magnetic device is not saturated and a second sub-zone having a higher magnetic field strength where the magnetization of soft-magnetic localization elements of the magnetic device is saturated are formed in the field of view,
changing the position in space of the two sub-zones in the field of view, by means of a magnetic drive field so that the magnetization of the soft-magnetic localization elements of the magnetic device changes locally,
changing the position in space of the field of view,
acquiring detection signals, which detection signals depend on the magnetization in the field of view, which magnetization is influenced by the change in the position in space of the first and second sub-zone,
processing said detection signals, and
controlling the generation of magnetic fields to move the field of view to a position such that the magnetic device is positioned between a target position of the magnetic device and the center of the field of view in order to generate a force for moving the magnetic device in the direction of the target position and to thereafter or simultaneously move the field of view to a position such that the magnetic device is positioned within the field of view for localizing the magnetic device.
